(19) United States
(12) Patent Application Publication
(10) Pub. No.: US 2006/0229609 A1
(43) Pub. Date: Oct. 12, 2006

(54) Microadjustment spinal joint fixture

(76) Inventor: Chao-Jan Wang, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/082,663

(22) Filed: Mar. 18, 2005

(51) Int. Cl. A61F 2/30

(52) U.S. Cl. 606/61

(57) ABSTRACT

A spinal joint fixture includes a first threaded fixture and a second threaded fixture respectively having multiple blind holes, a connection rod a first end of which is securely connected to a first end of the first threaded fixture and a second end of which is securely connected to a first end of the second threaded fixture and at least one universal joint sandwiched between the first end and second end of the connection rod such that the first threaded fixture is movable relative to the second threaded fixture.

### BACKGROUND OF THE INVENTION

1. Field of the Invention

The present invention relates to a spinal joint fixture, and more particularly to a spinal joint fixture which is able to be adjusted within a limited range to adapt to movement of the spinal joint.

2. Description of Related Art

Artificial spinal joint permits the spine to move within a limited range such that a patient wearing the spinal joint is able to move even though the patient's spine is injured. With reference to FIGS. 8 and 9, a conventional spinal joint fixture is shown and has a supporting rod (70) with two connecting heads (71) respectively and detachably connected to two ends of the supporting rod (70). Each connecting rod (71) has an extension hole (710) to allow an extension of one of the two ends of the supporting rod (70), a securing hole (711) laterally defined relative to and communicating with the extension hole (710) to allow a securing rod (72) to extend therethrough so as to fix the supporting rod (70) relative to the connecting head (71) after the supporting rod (70) is inserted into the extension hole (710) and a connecting hole (712) laterally defined relative to the extension hole (710) to allow an extension of a threaded fixture (73) to connect to a nut.

It is noted that when the conventional spinal joint fixture is used, the securing rod (72) is first screwed to the securing hole (711) to respectively secure each of the connecting heads (71) relative to the supporting rod (70). Then the threaded fixture (73) is extended through the connecting hole (712) and the nut is threadingly connected to an end of the threaded fixture (72) to fix the threaded fixture (72) on the connecting head (71). Thereafter, each of the two threaded fixtures (73) are respectively extended into an outer surface of a portion of two vertebrae to secure the spinal joint if needed.

After the employment of the spinal joint fixture, it is noted that the spinal joint is firmly fixed by the two threaded fixtures (73) so that when the spinal joint is responsible for a patient's bending movement, the patient wearing the spinal joint fixture is not able to bend due to the limitation of the supporting rod (70) and the connecting heads (71). Thus the patient's movement is confined and the healing time is prolonged.

To overcome the shortcomings, the present invention tends to provide an improved micro-adjustment spinal joint fixture to mitigate the aforementioned problems.

### SUMMARY OF THE INVENTION

The primary objective of the present invention is to provide a micro-adjustment spinal joint fixture to still allow the patient wearing the spinal joint fixture of the present invention to move appropriately.

In order to accomplish the objective, the spinal joint fixture of the present invention includes two threaded fixtures interconnected with one another via a connection rod with at least one universal joint provided between the two threaded fixtures so that the two threaded fixtures are able to move within a limited range.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of the spinal joint fixture of the present invention;

FIG. 2 is an exploded perspective view of the spinal joint fixture in FIG. 1;

FIG. 3 is a schematic view showing the application of the spinal joint fixture of the present invention on two adjacent vertebrae;

FIGS. 4 and 5 are schematic views in partial cross section to show that the spinal joint fixture of the present invention allows the spine to move within a limited range;

FIG. 6 is a schematic view in partial cross section to show that the spinal joint fixture of the present invention is located at a different position with respect to the position shown in FIGS. 4 and 5;

FIG. 7 is an exploded perspective view showing a different embodiment of the spinal joint fixture of the present invention;

FIG. 8 is an exploded perspective view of a conventional spinal joint fixture; and

FIG. 9 is a schematic view showing the application of the conventional spinal joint fixture of FIG. 8.

### DETAILED DESCRIPTION OF THE PREFERRED EMBODIMENT

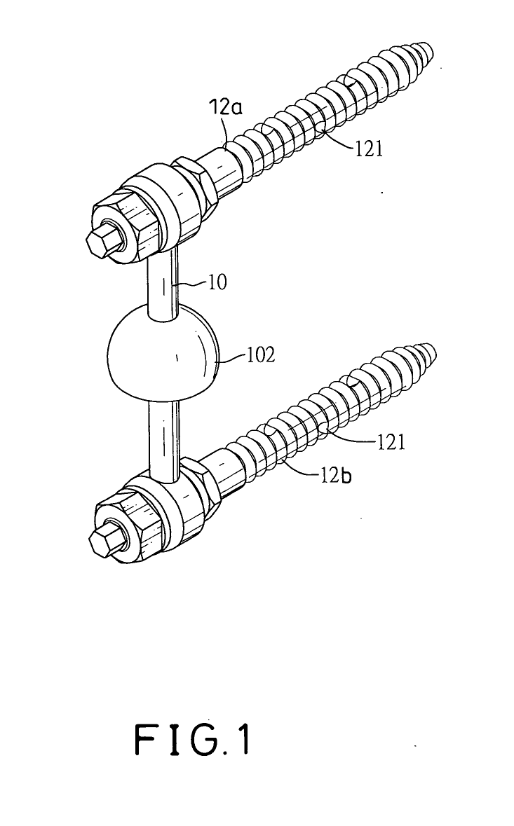

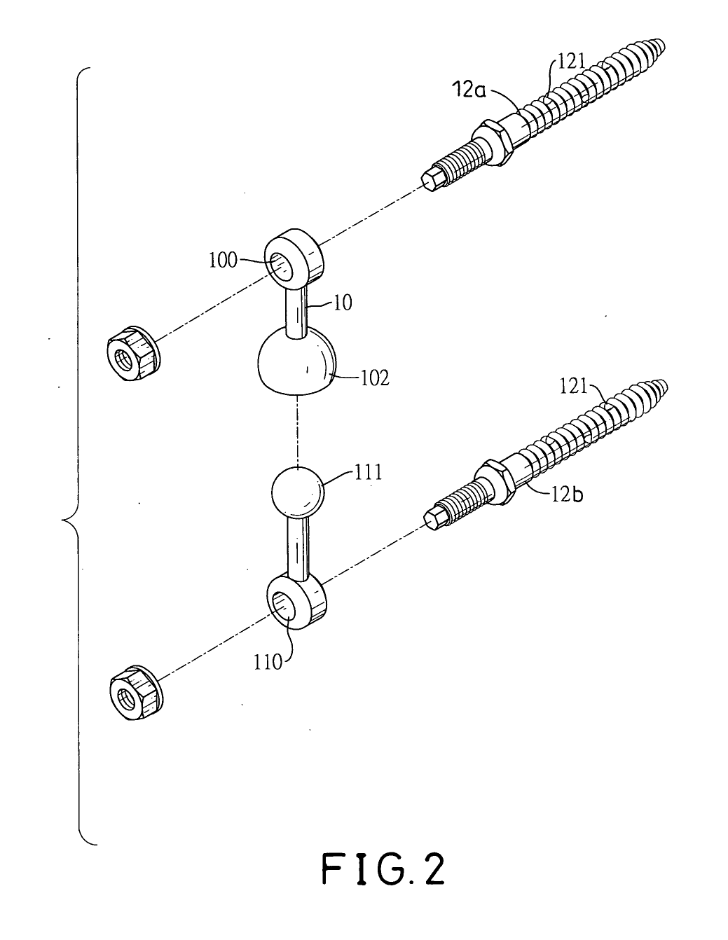

With reference to FIGS. 1 and 2, the spinal joint fixture in accordance with the present invention includes two threaded fixtures (12a,12b), a first threaded fixture (12a) and a second threaded fixture (12b), interconnected with one another via a connection rod (10) with at least one universal joint provided between the two threaded fixtures (12a,12b) so that the two threaded fixtures (12a,12b) are able to move within a limited range. Further, the two threaded fixtures (12a,12b) respectively have multiple blind holes (121) defined in an outer periphery of each of the two threaded fixtures (12a,12b) to allow fluid inside the spine to flow into the blind holes (121) so that the two threaded fixtures (12a,12b) are able to firmly fixed to the spine.

The connection rod (10) has a first end provided with a first through hole (100) corresponding to a first end of the first threaded fixture (12a) and a second end provided with a second through hole (110) corresponding to a first end of the second threaded fixture (12b). The connection rod (10) further has a bowl (102) and a sphere (111) movably received inside the bowl (102). How the connection rod (10) has the bowl (102) and the sphere (111) is described as follows. The connection rod (10) is divided into two sections, a first section and a second section, each having two ends. The first section has the first through hole (100) formed on a first end thereof and the bowl (102) formed on a second end thereof. The second section has the sphere (111) formed on a first end thereof and the second through hole (110) formed on a second end thereof. The sphere (111) is pivotally yet securely fitted into the bowl (102) and the interrelationship between the sphere (111) and the bowl (102) allows the sphere (111) to move inside the bowl (102). It is noted that an inner surface area of the bowl (102) is larger than an outer surface area of a half of the sphere (111) such that once the sphere (102) is received inside the bowl (102), the bowl (102) is able to hold the sphere (111) and the sphere (111) will not escape the bowl (102).

When the spinal joint fixture of the present invention is assembled, first ends of the first threaded fixture (12a) and the second threaded fixture (12b) are extended through the first through hole (100) and the second through hole (11) to respectively connect to a nut (not numbered). After the nuts are threadingly connected to the first ends of the first threaded fixture (12a) and the second threaded fixture (12b), the assembly of the spinal joint fixture of the present invention is finished and is presented as the structure shown in FIG. 1.

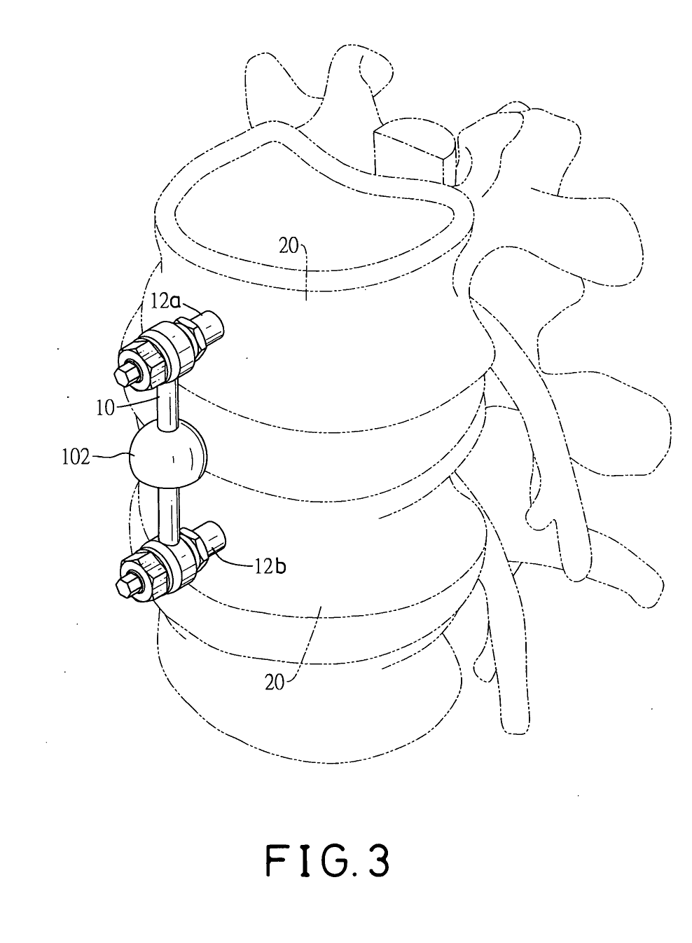

With reference to FIG. 3, it is noted that the spinal joint fixture of the present invention is applied to two adjacent vertebrae (20) via extension of the second ends of the first threaded fixture (12a) and the second threaded fixture (12b) into the two adjacent vertebral columns (20).

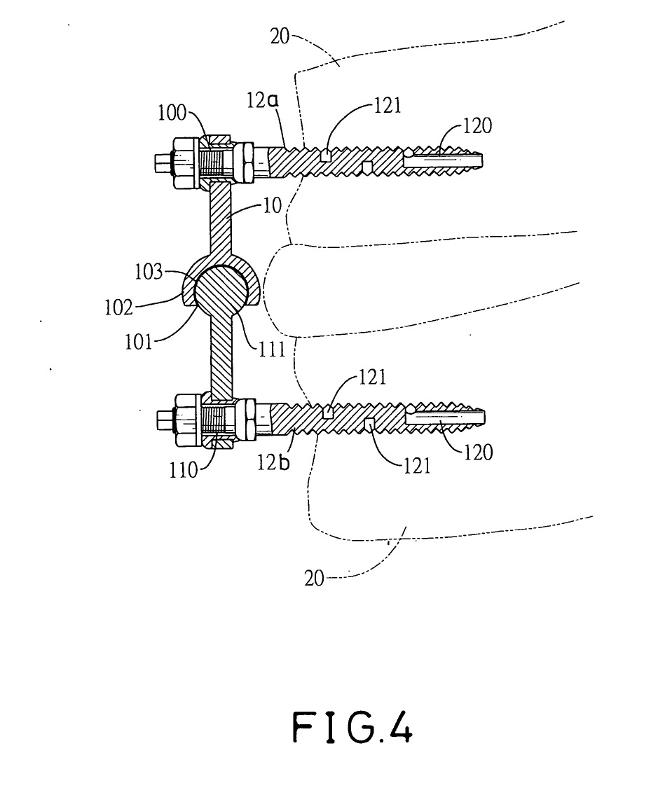

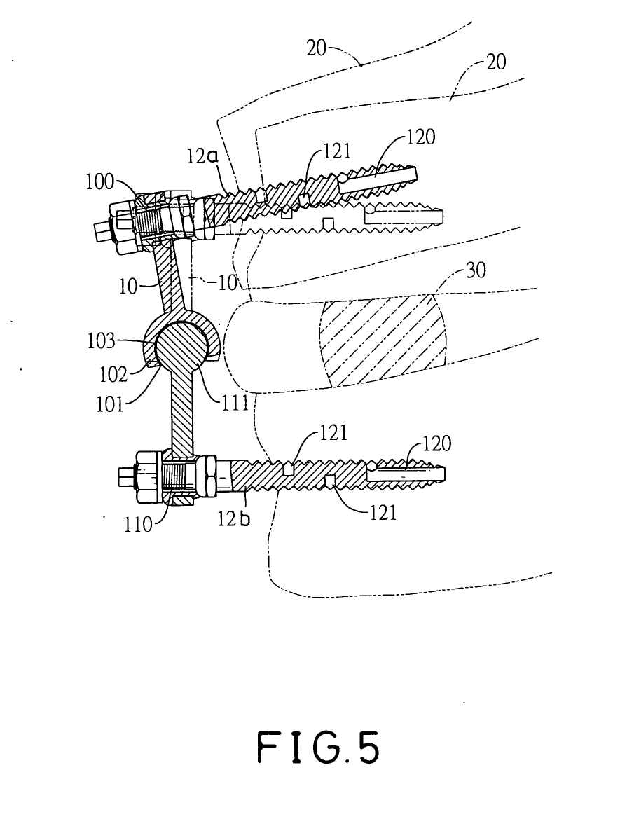

With reference to FIGS. 4 and 5, after the spinal joint fixture is fixed to the two adjacent vertebral columns (20), an inter-vertebral disk (30) sandwiched between the two adjacent vertebral columns (20) will be protected from pressure resulted from movement of the two adjacent vertebral columns (20). That is, due to the freely moveable feature of the sphere (111) inside the bowl (102), the first threaded fixture (12a) is able to move relative to the second threaded fixture (12b) so as to adapt to movement of a patient suffering from spinal injury. Furthermore, a padding (103) may be added to the inner periphery of the bowl (102). The padding (103) may be a coating or a thin film of a suitable lubricant. In addition to the blind holes (121), the two threaded fixtures (12a,12b) have channels (120) defined through the outer periphery of each of the two threaded fixtures (12a,12b) to receive therein the fluid in the spine.

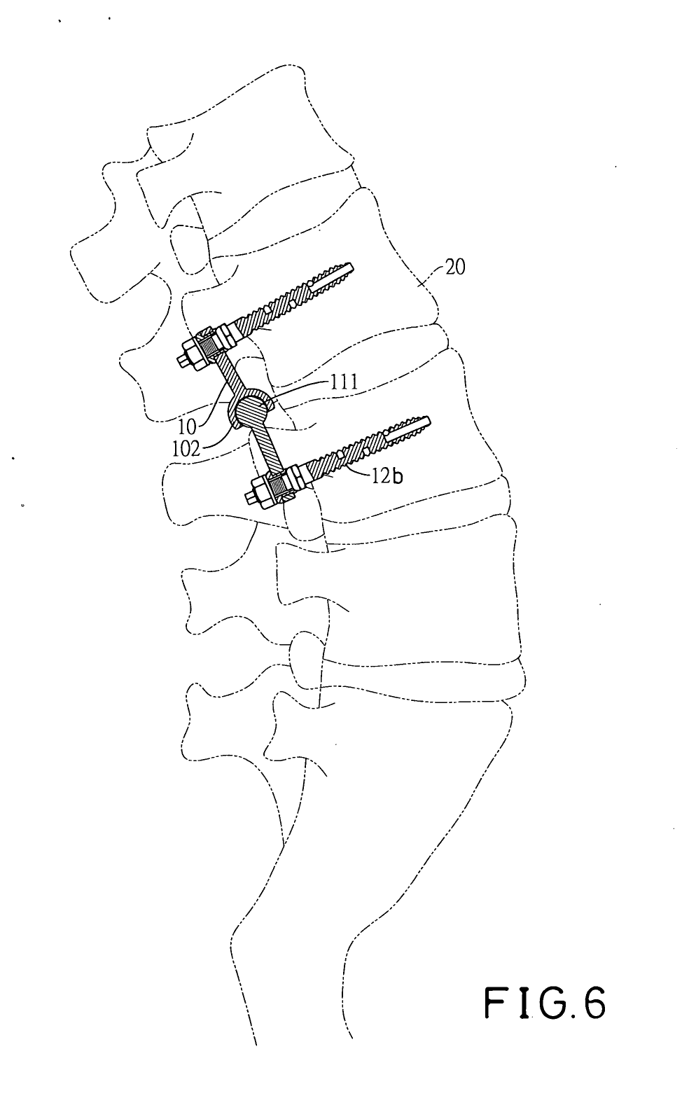

With reference to FIG. 6, it is noted that the spinal joint fixture may be applied to a different position in the spine with respect to the position as shown in FIGS. 4 and 5.

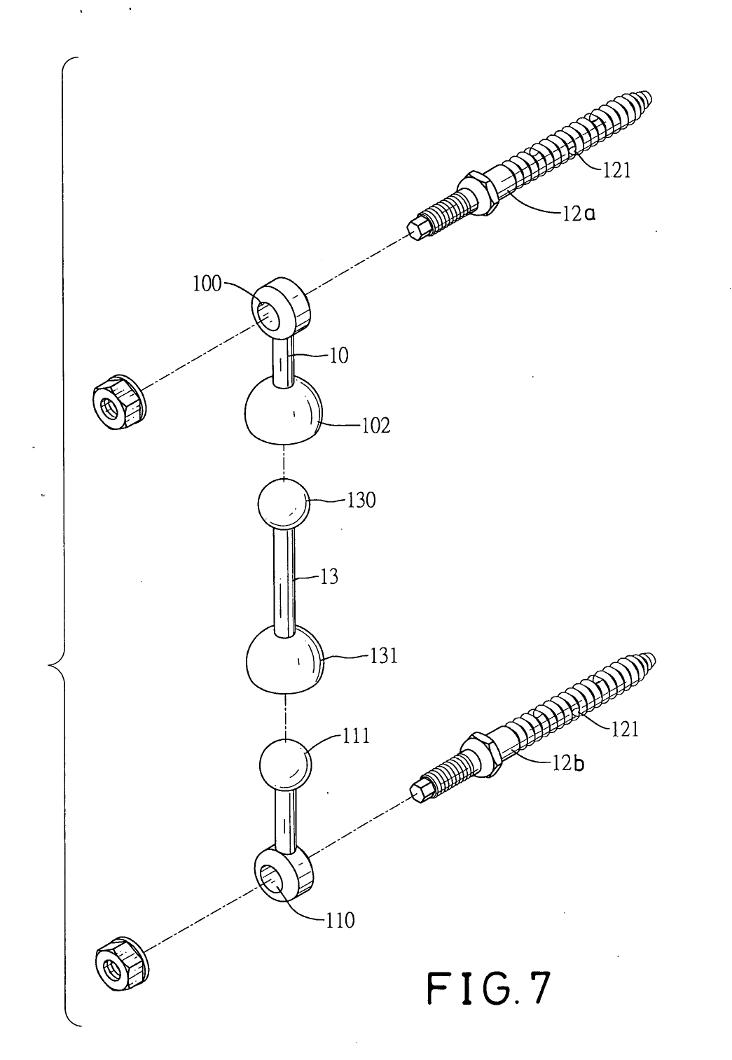

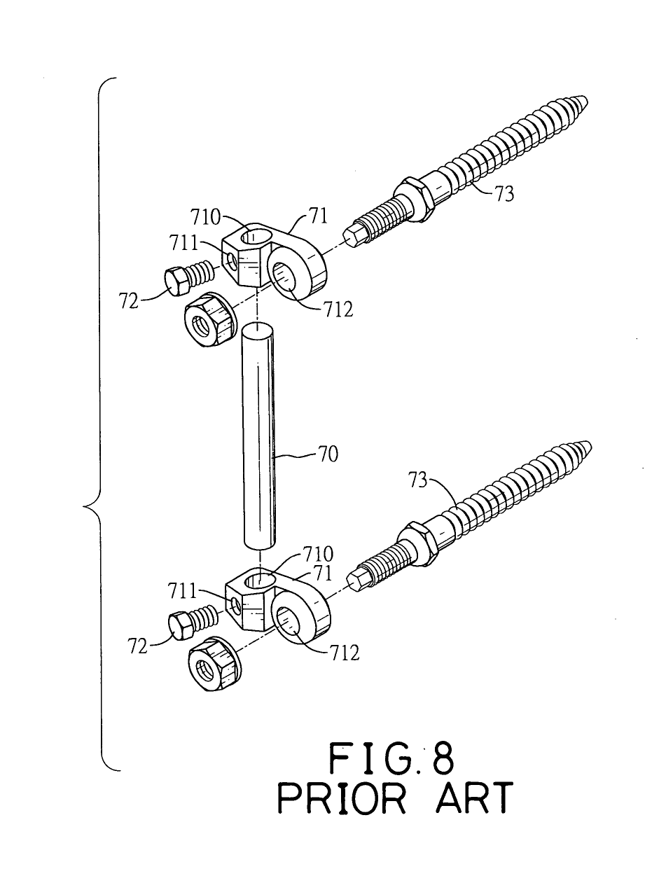

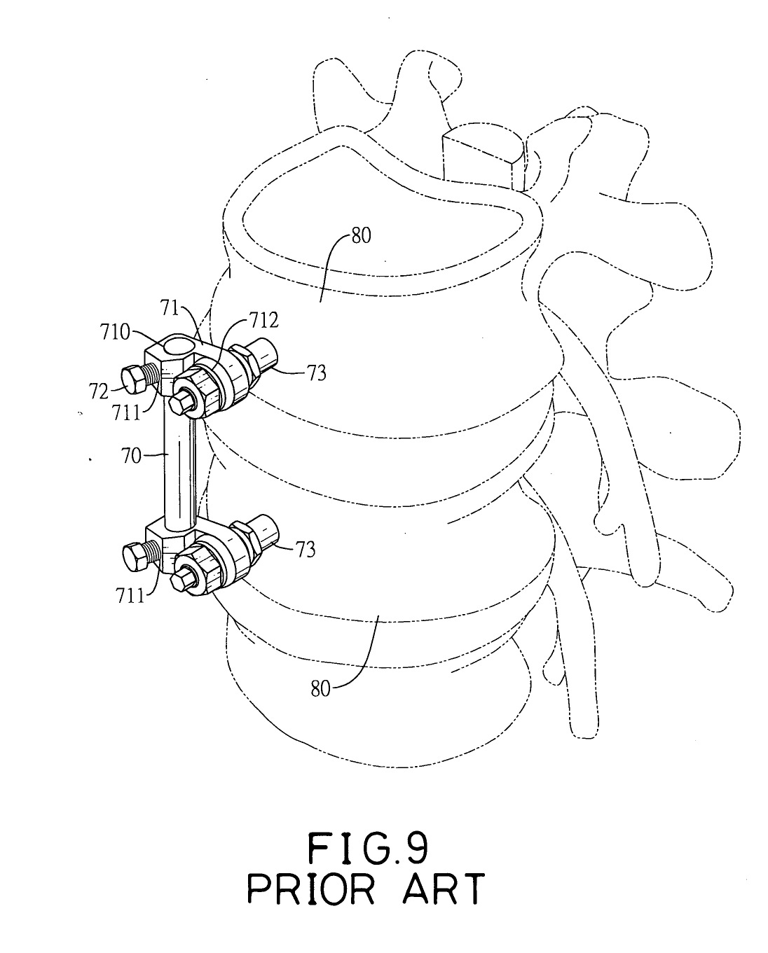

With reference to FIG. 7, an auxiliary rod (13) may be added between the first section and the second section of the connection rod (10). The auxiliary rod (13) is provided with a second sphere (130) formed on a first end thereof to correspond to the bowl (102) of the first section of the connection rod (10) and a second bowl (131) formed on a second end thereof to correspond to the sphere (111) of the second section of the connection rod (10). That is, the second sphere (130) is interference fitted into the bowl (102) and the sphere (111) is interference fitted into the second bowl (131). Thus an overall length of the connection rod (10) is lengthened and an increased range of travel is provided by the doubling of pivots. Thus, the user is able to use the spinal joint fixture of this kind to a large range of area in the spine of a patient.

It is noted that due to the interrelationship between the bowl and the sphere, the patient suffering from spinal injury and having an inter vertebral disk replaced with an artificial disk can still move within a limited range to allow the muscle surrounding the new disk to grow strong such that the healing duration is reduced.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A spinal joint fixture comprising:
a first threaded fixture and a second threaded fixture respectively having multiple blind holes and channels defined through an outer periphery of each of the first threaded fixture and the second threaded fixture;
a connection rod, a first end of which is securely connected to a first end of the first threaded fixture and a second end of which is securely connected to a first end of the second threaded fixture; and
at least one universal joint sandwiched between the first end and second end of the connection rod such that the first threaded fixture is movable relative to the second threaded fixture.

2. The spinal joint fixture as claimed in claim 1, wherein the first end of the connection rod is provided with a first through hole to allow an extension of the first end of the first threaded fixture for engagement with a first nut and the second end of the connection rod is provided with a second through hole to allow an extension of the second end of the second threaded fixture for engagement with a second nut.

3. The spinal joint fixture as claimed in claim 1, wherein the universal joint includes a bowl and a sphere movably received in the bowl.

4. The spinal joint fixture as claimed in claim 2, wherein the universal joint includes a bowl and a sphere movably received in the bowl.

5. The spinal joint fixture as claimed in claim 3, wherein the connection rod is divided into a first section having the first through hole formed on a first end thereof and the bowl formed on a second end thereof and a second section having the sphere formed on a first end thereof and the second through hole formed on a second end thereof.

6. The spinal joint fixture as claimed in claim 4, wherein the connection rod is divided into a first section having the first through hole formed on a first end thereof and the bowl formed on a second end thereof and a second section having the sphere formed on a first end thereof and the second through hole formed on a second end thereof.

7. The spinal joint fixture as claimed in claim 5 further comprising an auxiliary rod sandwiched between the first section and the second section of the connection rod, the auxiliary rod having a first end provided with a second sphere movably received in the bowl and a second end provided with a second bowl to movably receive therein the sphere so as to lengthen the connection rod.

8. The spinal joint fixture as claimed in claim 6 further comprising an auxiliary rod sandwiched between the first section and the second section of the connection rod, the auxiliary rod having a first end provided with a second sphere movably received in the bowl and a second end provided with a second bowl to movably receive therein the sphere so as to lengthen the connection rod.

9. A spinal joint fixture comprising:
a first threaded fixture and a second threaded fixture respectively having multiple blind holes and channels defined through an outer periphery of each of the first threaded fixture and the second threaded fixture;
a connection rod, a first end of which is securely connected to a first end of the first threaded fixture and a second end of which is securely connected to a first end of the second threaded fixture, wherein the first end of the connection rod is provided with a first through hole to allow an extension of the first end of the first threaded fixture for engagement with a first nut and the second end of the connection rod is provided with a second through hole to allow an extension of the second end of the second threaded fixture for engagement with a second nut, wherein the connection rod is divided into a first section having the first through hole formed on a first end thereof and the bowl formed on a second end thereof and a second section having the sphere formed on a first end thereof and the second through hole formed on a second end thereof; and
at least one universal joint sandwiched between the first end and second end of the connection rod and including a bowl and a sphere movably received in the bowl such that the first threaded fixture is movable relative to the second threaded fixture.

10. A spinal joint fixture comprising:
a first threaded fixture and a second threaded fixture respectively having multiple blind holes and channels defined through an outer periphery of each of the first threaded fixture and the second threaded fixture;
a connection rod, a first end of which is securely connected to a first end of the first threaded fixture and a second end of which is securely connected to a first end of the second threaded fixture, wherein the first end of the connection rod is provided with a first through hole to allow an extension of the first end of the first threaded fixture for engagement with a first nut and the second end of the connection rod is provided with a second through hole to allow an extension of the second end of the second threaded fixture for engagement with a second nut, wherein the connection rod is divided into a first section having the first through hole formed on a first end thereof and the bowl formed on a second end thereof and a second section having the sphere formed on a first end thereof and the second through hole formed on a second end thereof;
at least one universal joint sandwiched between the first end and second end of the connection rod and including a bowl and a sphere movably received in the bowl such that the first threaded fixture is movable relative to the second threaded fixture; and
an auxiliary rod sandwiched between the first section and the second section of the connection rod, the auxiliary rod having a first end provided with a second sphere movably received in the bowl and a second end provided with a second bowl to movably receive therein the sphere so as to lengthen the connection rod.
